(19) United States
(12) Patent Application Publication
(10) Pub. No.: US 2026/0250348 A1
(43) Pub. Date: Aug. 27, 2026

(54) PREPARATION METHOD AND APPLICATION OF sCRA1 MUNTANT NANOPARTICLE AGAINST CV-B3 INFECTION

(71) Applicant: Institute of Medical Biology, Chinese Academy of Medical Sciences, Kunming (CN)

(72) Inventors: LONGDING LIU, Kunming (CN); XINGLONG ZHANG, Kunming (CN); HUIWEN ZHENG, Kunming (CN); HENG LI, Kunming (CN); YUN LIAO, Kunming (CN); JINGJING WANG, Kunming (CN); XIN ZHAO, Kunming (CN); DANDAN LI, Kunming (CN); LI YU, Kunming (CN); HENG ZHAO, Kunming (CN); HAIJING SHI, Kunming (CN)

(21) Appl. No.: 19/275,158

(22) Filed: Jul. 21, 2025

(30) Foreign Application Priority Data
Feb. 21, 2025 (CN) 2025101921721

(51) Int. Cl. C07K 14/705; A61K 38/00

(52) U.S. Cl. CPC C07K 14/70503; A61K 38/00

(57) ABSTRACT

A preparation method of an application of a sCAR1 mutant nanoparticle against CV-B3 infection are provided, thereby solving a problem of the current lack of preventive vaccines and specific therapeutic drugs for the CV-B3 infection. A sCAR1 Mu+Ferritin nanoparticle that fully exhibits sCAR1 Mu is constructed, and IC₅₀ values of the sCAR1 Mu+Ferritin nanoparticle against the CV-B3 infection in Vero cells and SH-SY5Y cells are 413.05 ng/mL and 140.93 ng/mL, respectively. When 200 μg of sCAR1 Mu+Ferritin is injected intraperitoneally 6 hours prior to an intraperitoneal injection of a lethal dose of CV-B3, it significantly reduces viral loads in liver, spleen, brain, and heart tissues, effectively alleviates inflammatory pathology in heart and brain tissues, and provides a 68.75% survival rate.

### CROSS-REFERENCE TO RELATED APPLICATION

This application claims priority to Chinese Patent Application No. 202510192172.1, filed on Feb. 21, 2025, which is herein incorporated by reference in its entirety.

### TECHNICAL FIELD

The disclosure relates to the technical field of biomedicines, and more particularly to a preparation method and an application of a soluble coxsackievirus and adenovirus receptor 1 (sCAR1) mutant nanoparticle against coxsackievirus B3 (CV-B3) infection.

### STATEMENT REGARDING SEQUENCE LISTING

The sequence listing associated with this application is provided in text format in lieu of a paper copy and is hereby incorporated by reference into the specification. The name of the XML file containing the sequence listing is 25037TBYX-USP1-MF-2025-0036-SL.xml. The XML file is 8,627 bytes; is created on Jul. 7, 2025; and is being submitted electronically via patent center.

### BACKGROUND

CV-B3 is a symmetrical particle with a regular icosahedron and no envelope structure in viral morphology with a diameter of approximately 30 nanometers (nm). A capsid of the CV-B3 is composed of four structural proteins: virus protein 1 (VP1), virus protein 2 (VP2), virus protein 3 (VP3), and virus protein 4 (VP4). Among them, the VP1, the VP2, and the VP3 are displayed outside the capsid of the CV-B3, while the VP4 is enclosed inside the capsid of the CV-B3. The Can you region formed by the VP1, the VP2, and the VP3 is a crucial region for the binding of CV-B3 particles to a coxsackievirus and adenovirus receptor (CAR). CV-B3 infection can cause clinical symptoms such as hand-foot-mouth disease-like rashes, blisters, viral myocarditis, and viral meningitis. The viral meningitis and the viral myocarditis usually cause more severe pathological symptoms in the body and may lead to fatalities in some patients, particularly infected infants and young children. At present, there are no prevention vaccines or specific antiviral drugs available for the CV-B3 infection. Therefore, clinical treatment of the CV-B3 infection relies solely on supportive therapy. Research on antiviral drugs primarily focuses on key biochemical and molecular biological events occurring during a viral infection cycle, including viral attachment and binding, viral genome release, viral genome replication, formation of a viral replication microenvironment, translation and synthesis of viral proteins, enzymatic properties of viral non-structural proteins, and assembly and release of viral particles.

When a virus is pandemic or a viral strain undergoes high-frequency mutations, dual delays in both vaccine development and efficacy onset makes it difficult to provide effective measures for epidemic prevention and control in the first place. The development of specific neutralizing antibodies may face dilemmas of high technical barriers and reduced or even invalid neutralizing potency of antibodies due to viral mutations. Small-molecule interfering drugs face problems such as difficult mechanism identification, large screening difficulty, low delivery efficiency, and complex pharmacokinetics.

### SUMMARY

A purpose of the disclosure is to provide a preparation method and an application of a sCAR1 mutant nanoparticle against CV-B3 infection, thereby solving a problem of the current lack of preventive vaccines and specific therapeutic drugs for the CV-B3 infection.

To achieve the above purpose, the disclosure provides following technical solutions.

The disclosure provides an application of a sCAR1 protein, including: preparing a drug against CV-B3 by using the sCAR1 protein. The sCAR1 protein is a splice variant member coxsackievirus and adenovirus receptor 1 (CAR1) protein of a CAR protein containing an intact extracellular segment (D1 and D2 domains), and the amino acid sequence of the sCAR1 protein is shown in SEQ ID NO: 2.

The disclosure provides a modified soluble coxsackievirus and adenovirus receptor 1 mutant (sCAR1 Mu) protein, and the modified sCAR1 Mu protein is obtained by modifying the sCAR1 protein with the amino acid sequence as shown in SEQ ID NO: 2. The amino acid sequence of the modified sCAR1 Mu protein is shown in SEQ ID NO: 5.

The disclosure provides a mutant nanoparticle. The mutant nanoparticle has a ferritin nanoparticle as a matrix and the modified sCAR1 Mu protein as described above displayed on a surface of the ferritin nanoparticle.

The disclosure provides a mutant nanoparticle, and the amino acid sequence of the mutant nanoparticle is shown in SEQ ID NO: 1.

The disclosure provides a recombinant expression vector for expressing the mutant nanoparticle as described above, and a base vector of the recombinant expression vector is pTT5.

The disclosure provides an application of the modified sCAR1 Mu protein, the mutant nanoparticle, or the recombinant expression vector as described above, including: preparing a drug against CV-B3 by using the modified sCAR1 Mu protein, the mutant nanoparticles, or the recombinant expression vector.

The disclosure provides a modified coxsackievirus and adenovirus receptor 3 mutant (CAR3 Mu) protein, and the modified CAR3 Mu protein is obtained by modifying a CAR protein. The amino acid sequence of the modified CAR3 Mu protein is shown in SEQ ID NO: 6.

In the disclosure, both the modified CAR3 Mu protein and the modified sCAR1 Mu protein are based on modification of the CAR protein. During this modification, amino acid residues that have strong interactions with the CV-B3 are retained, while sites that have strong interactions with the CAR protein itself and a junctional adhesion molecule-like (JAML) protein are mutated. The above modified mutants (i.e., the modified CAR3 Mu protein and the modified sCAR1 Mu protein) are both based on a same inventive concept and both are used against the CV-B3.

The disclosure provides a mutant nanoparticle. The mutant nanoparticle has a ferritin nanoparticle as a matrix and the modified CAR3 Mu protein as described above displayed on a surface of the ferritin nanoparticle.

The disclosure provides a mutant nanoparticle, and the amino acid sequence of the mutant nanoparticle is shown in SEQ ID NO: 7.

The disclosure provides an application of the modified CAR3 Mu protein or the mutant nanoparticle as described above, including: preparing a drug against CV-B3 by using the modified CAR3 Mu protein or the mutant nanoparticles.

The disclosure may achieve the following beneficial effects.

1. Based on a principle of receptor analog-based antiviral activity, the disclosure discloses that the CAR1 protein, a splice variant member of the CAR protein containing the intact extracellular segment (D1 and D2 domains), has an antiviral effect against the CV-B3 infection in vitro. By binding to the CV-B3, a capsid of the CV-B3 is shed and a viral genome is released, thereby limiting the infectivity of the CV-B3.

2. The CAR protein has a characteristic of forming intramolecular homodimers and can bind to cell membrane surface molecules CAR and JAML, which may pose a potential risk of promoting cell proliferation. To overcome this risk, the disclosure designs the modified mutant sCAR1 Mu, which no longer forms intramolecular homodimers and does not bind to the cell membrane surface molecules CAR and JAML. This significantly improves the efficacy and safety of the modified mutant sCAR1 Mu against the CV-B3 infection.

3. Based on a Ferritin protein, a sCAR1 Mu+Ferritin nanoparticle that fully displays the modified sCAR1 Mu protein is constructed. Half maximal inhibitory concentration (IC₅₀) values for blocking the CV-B3 infection in Vero and SH-SY5Y cells are 413.05 nanograms per milliliter (ng/mL) and 140.93 ng/mL, respectively.

4. The efficacy and safety of sCAR1 Mu+Ferritin against the CV-B3 infection are further evaluated in 3-day-old Balb/c mice. The disclosure discloses that an intraperitoneal injection of 200 micrograms (g) sCAR1 Mu+Ferritin 6 hours in advance, followed by an intraperitoneal injection of a lethal infectious dose of the CV-B3, i.e., a half maximal cell culture infectious dose (CCID₅₀) of the CV-B3 of 10^{3.5}, significantly reduces viral loads in liver, spleen, brain, and heart tissues, effectively alleviates inflammatory pathology in heart and brain tissues, and provides a survival rate of 68.75%.

### BRIEF DESCRIPTION OF DRAWINGS

FIG. 1 illustrates a schematic diagram of expression constructs of sCAR1 and CAR3, and their respective structure predictions generated by using SWISS-MODLE. Specifically, in FIG. 1, UniProt represents the Universal Protein Resource.

FIG. 2 illustrates a schematic diagram of an analysis of eukaryotic expression and purification of sCAR1/CAR3. Specifically, in FIG. 2, A illustrates a Coomassie blue staining analysis of expression of the sCAR1/CAR3 following transfection of plasmids into 293T cells; and B illustrates Western Blot identification of the sCAR1/CAR3 after expression and purification.

FIGS. 3A-3D illustrate schematic diagrams of interaction between the sCAR1/CAR3 and CV-B3, leading to uncoating of the CV-B3 and release of a viral genome. Specifically, FIG. 3A illustrates a schematic diagram of ultracentrifugation of a CV-B3 viral lysate to obtain intact CV-B3 virions. FIG. 3B illustrates a schematic diagram of immunoprecipitation occurs between the sCAR1/CAR3 and the CV-B3. FIG. 3C illustrates a schematic diagram of co-incubation of the sCAR1/CAR3 with the CV-B3 at room temperature, resulting in the release of the viral genome and transforming the CV-B3 into empty capsids. FIG. 3D illustrates a schematic diagram of co-incubation of CV-B3 with varying concentrations of sCAR1/CAR3, ranging from a high concentration of 500 micrograms per milliliter (g/mL) to a low concentration of 16 g/mL, promoting the release of the viral genome from the CV-B3 into a supernatant.

FIG. 4 illustrates a schematic diagram of an analysis of potential safety issues associated with the sCAR1/CAR3. Specifically, in FIG. 4, A illustrates intramolecular dimerization of the sCAR1/CAR3; B illustrates immunoprecipitation of the sCAR1/CAR3 with cell membrane surface adhesion molecules CAR and JAML; C illustrates abnormal cell proliferation of high concentrations (400 g/mL) of the sCAR1/CAR3; and D illustrates an analysis of effects of the high concentrations (400 g/mL) of the sCAR1/CAR3 on cell adhesion and pro-inflammatory cytokines. In addition, in FIG. 4, ns represents no significant.

FIG. 5 illustrates a schematic diagram of an analysis of expression and purification of sCAR1 Mu and CAR3 Mu. Specifically, in FIG. 5, A illustrates a Coomassie blue staining analysis of a supernatant after eukaryotic expression in 293T cells, flow-through after purification, wash fraction, and eluate; and B illustrates Western Blot identification of the sCAR1 Mu and the CAR3 Mu.

FIG. 6 illustrates a schematic diagram of a safety analysis of the sCAR1 Mu and the CAR3 Mu. Specifically, in FIG. 6, A illustrates detection of intramolecular dimerization of the sCAR1 Mu and the CAR3 Mu after bis(sulfosuccinimidyl) suberate (BS³) cross-linking; B illustrates immunoprecipitation detection of sCAR1 Mu/CAR3 Mu and the cell membrane surface adhesion molecules CAR and JAML; C illustrates effects of various concentrations of the sCAR1 Mu/CAR3 Mu on proliferation of Vero and SH-SY5Y cells as determined by a cell counting kit-8 (CCK8) assay; and D illustrates an analysis of impact of a high concentration (400 g/mL) of the sCAR1 Mu/CAR3 Mu on cell proliferation and message ribonucleic acid (mRNA) levels of pro-inflammatory cytokines in the 293T cells.

FIG. 7 illustrates a schematic diagram of detection and comparison of antiviral activity against CV-B3 infection at a cellular level for both sCAR1/CAR3 and their mutants sCAR1 Mu/CAR3 Mu.

FIG. 8 illustrates a schematic diagram of structural predictions by using SWISS-MODEL and expression and purification analysis of sCAR1 Mu+Ferritin and CAR3 Mu+Ferritin. Specifically, in FIG. 8, A illustrates expression plasmid constructions and structural predictions by using SWISS-MODEL for the sCAR1 Mu+Ferritin and the CAR3 Mu+Ferritin; B illustrates a Coomassie blue staining analysis of a supernatant after expression, flow-through after purification, wash fraction, and eluate for the sCAR1 Mu+Ferritin and the CAR3 Mu+Ferritin; and C illustrates Western Blot identification of the sCAR1 Mu+Ferritin and the CAR3 Mu+Ferritin after purification.

FIG. 9 illustrates a schematic diagram of morphological characterizations of the sCAR1 Mu+Ferritin and the CAR3 Mu+Ferritin. Specifically, in FIG. 9, A illustrates native polyacrylamide gel electrophoresis (PAGE) Coomassie blue staining of the sCAR1 Mu+Ferritin and the CAR3 Mu+Ferritin; B illustrates transmission electron microscopy images of the sCAR1 Mu+Ferritin and the CAR3 Mu+Ferritin; and C illustrates detection of intramolecular dimerization of the sCAR1 Mu+Ferritin and the CAR3 Mu+Ferritin after BS³ crosslinking.

FIG. 10 illustrates a schematic diagram of detection and comparison of antiviral capabilities against CV-B3 infection at the cellular level for both the sCAR1 Mu/CAR3 Mu and the sCAR1 Mu+Ferritin/CAR3 Mu+Ferritin.

FIGS. 11A-11I illustrate schematic diagrams of antiviral effects of the sCAR1 Mu+Ferritin in vivo in mice. Specifically, FIG. 11A illustrates a schematic diagram of changes in survival rate of mice infected with different doses of the CV-B3. FIG. 11B illustrates a schematic diagram of changes in body weight of mice infected with the CV-B3 subjected to intervention with different doses of the sCAR1 Mu+Ferritin. FIG. 11C illustrates a schematic diagram of survival curves of the mice infected with the CV-B3 subjected to intervention with the different doses of the sCAR1 Mu+Ferritin. FIGS. 11D-11G illustrate schematic diagrams of comparison of viral load changes in various tissues and organs subjected to intervention with the sCAR1 Mu+Ferritin during the CV-B3 infection. FIG. 11H illustrates a schematic diagram of inflammatory pathology in heart tissue subjected to intervention with the sCAR1 Mu+Ferritin during the CV-B3 infection. FIG. 11I illustrates a schematic diagram of inflammatory pathology in brain tissue subjected to intervention with the sCAR1 Mu+Ferritin during the CV-B3 infection.

FIG. 12 illustrates a schematic diagram of expression and purification of a CAR3-6 histidine (6His)-pTT5 plasmid. Specifically, in FIG. 12, A illustrates that a 6His purification tag is linked to a C-terminus of the CAR3 via a four glycine and one serine (4GS) flexible linker, along with the sequence information of a native signal peptide of the CAR3. B illustrates an analysis of hydrophobicity of the native signal peptide of the CAR3. C illustrates Coomassie blue staining of a supernatant after purification of samples collected following expression of the CAR3-6His-pTT5 plasmid. D illustrates Western Blot identification of purified samples from both a cell lysate and the supernatant following expression of the CAR3-6His-PTT5 plasmid.

FIG. 13 illustrates a schematic diagram of expression and purification of a CAR3 (with a more hydrophilic signal peptide replacing its native signal peptide)-10 histidine (10His)-pTT5 plasmid. Specifically, in FIG. 13, A illustrates that a 10His tag is attached to the C-terminus of the CAR3 via the 4GS flexible linker, along with the sequence information of the more hydrophilic signal peptide. B illustrates an analysis of hydrophobicity of the more hydrophilic signal peptide. C illustrates Coomassie blue staining of samples collected after expression and purification of the CAR3 (with a more hydrophilic signal peptide replacing its native signal peptide)-10His-PTT5 plasmid. D illustrates Western Blot identification of the samples collected after expression and purification of the CAR3 (with a more hydrophilic signal peptide replacing its native signal peptide)-10His-PTT5 plasmid.

### DETAILED DESCRIPTION OF EMBODIMENTS

The following provides a detailed description of technical solutions provided by the disclosure in conjunction with embodiments, but they should not be understood as limiting the scope of protection of the disclosure.

Sequence information involved in embodiments of the disclosure is as follows.

A recombinant protein, named sCAR1 Mu+Ferritin, is a fusion of two components: sCAR1 Mu, which is a mutant form of a CAR protein originating from the house mouse (Mus musculus) with a corresponding Gene identification number (ID) 13052, and a Human Ferritin Heavy chain, identified by a Gene ID 2495. This recombinant protein is designed for expression using a PTT5 plasmid vector.

The modified sequence, i.e., the amino acid sequence of sCAR1 Mu+Ferritin is as follows:
MGWSCIILFLVATGVHSLSITTPEQRIEKAKGETAYLPCKFTLSPEDQGP LAIAWLISPSDNQIVDQAIIAYSGDKIYDNAAPDLKGRVHFTSNDVKSGDASIQ VTNLQLSDIGTYQCAVKKAPGVANKKFLLTVLVKPSGTRCFVDGSEEIGNDFK LKCEPKEGSLPLQFEWQKLSDSQTMPTPWLAEMTSPVISVKQASSEYSGTYSC TVQNRVGSDQCMLRLDVVPGGGGSGGGGSGGGGSMTTASTSQVRQNYHQD SEAAINRQINLELYASYVYLSMSYYFDRDDVALKNFAKYFLHQSHEEREHAEK LMKLQNQRGGRIFLQDIKKPDCDDWESGLNAMECALHLEKNVNQSLLELHK LATDKNDPHLCDFIETHYLNEQVKAIKELGDHVTNLRKMGAPESGLAEYLFD KHTLGDSDNESGGGGSGGGGSGGGGSHHHHHHHHHH, as shown in SEQ ID NO: 1.

The amino acid sequence of extracellular domain of sCAR1 before mutation is as follows:
MARLLCFVLLCGIADFTSGLSITTPEQRIEKAKGETAYLPCKFTLSPED QGPLDIEWLISPSDNQIVDQVIILYSGDKIYDNYYPDLKGRVHFTSNDVKSGDA SINVTNLQLSDIGTYQCKVKKAPGVANKKFLLTVLVKPSGTRCFVDGSEEIGN DFKLKCEPKEGSLPLQFEWQKLSDSQTMPTPWLAEMTSPVISVKNASSEYSGT YSCTVQNRVGSDQCMLRLDVVP, as shown in SEQ ID NO: 2.

The amino acid sequence of a signal peptide is as follows:
MARLLCFVLLCGIADFTSG, as shown in SEQ ID NO: 3.

The amino acid sequence of a signal peptide is as follows:
MGWSCIILFLVATGVHS, as shown in SEQ ID NO: 4.

The sequence after signal peptide modification and amino acid point mutation is as follows:
MGWSCIILFLVATGVHSLSITTPEQRIEKAKGETAYLPCKFTLSPEDQGP LAIAWLISPSDNQIVDQAIIAYSGDKIYDNAAPDLKGRVHFTSNDVKSGDASIQ VTNLQLSDIGTYQCAVKKAPGVANKKFLLTVLVKPSGTRCFVDGSEEIGNDFK LKCEPKEGSLPLQFEWQKLSDSQTMPTPWLAEMTSPVISVKQASSEYSGTYSC TVQNRVGSDQCMLRLDVVP, as shown in SEQ ID NO: 5.

The amino acid sequence of a CAR3 Mu protein is as follows:
MGWSCIILFLVATGVHSLSITTPEQRIEKAKGETAYLPCKFTLSPEDQGP LAIAWLISPSDNQIVDQAIIAYSGDKIYDNAAPDLKGRVHFTSNDVKSGDASIQ VTNLQLSDIGTYQCAVKKAPGVANKKFLLTVLGKSSFLLSTGVEWGGGAELQ GGREGG, as shown in SEQ ID NO: 6.

The amino acid sequence of CAR3 Mu+Ferritin is as follows:
MGWSCIILFLVATGVHSLSITTPEQRIEKAKGETAYLPCKFTLSPEDQGP LAIAWLISPSDNQIVDQAIIAYSGDKIYDNAAPDLKGRVHFTSNDVKSGDASIQ VTNLQLSDIGTYQCAVKKAPGVANKKFLLTVLGKSSFLLSTGVEWGGGAELQ GGREGGGGGGSGGGGSGGGGSMTTASTSQVRQNYHQDSEAAINRQINLELYA SYVYLSMSYYFDRDDVALKNFAKYFLHQSHEEREHAEKLMKLQNQRGGRIF LQDIKKPDCDDWESGLNAMECALHLEKNVNQSLLELHKLATDKNDPHLCDFI ETHYLNEQVKAIKELGDHVTNLRKMGAPESGLAEYLFDKHTLGDSDNESGG GGSGGGGSGGGGSHHHHHIHHHH, as shown in SEQ ID NO:7.

### Embodiment

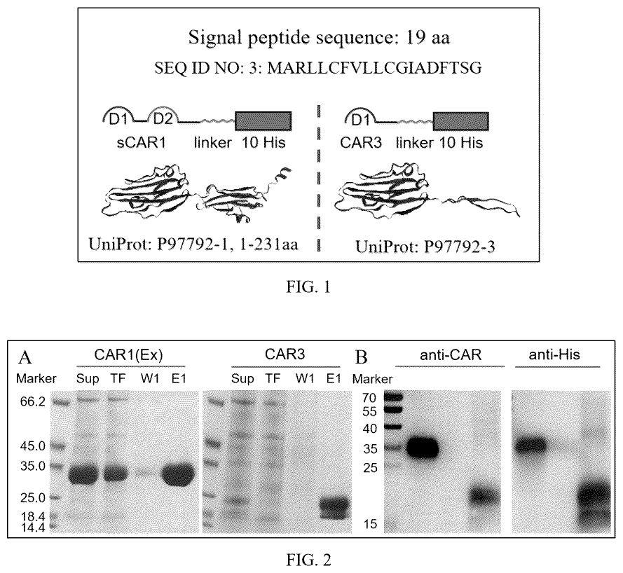

1, sCAR1 binds to CV-B3 and releases a viral genome to form non-infectious empty capsids. Specifically, A binding region of the CV-B3 with a CAR protein is mainly the D1 domain. In the disclosure, a plasmid is designed to connect a 10 His tag to a C-terminus of the sCAR1 and a C-terminus of CAR3 separately via a 4GS flexible linker. Structural predictions by using SWISS-MODEL illustrate that a recombinant sCAR1 can form the D1 and D2 domains and a recombinant CAR3 can form the D1 domain (as shown in FIG. 1).

After transfection and expression in 293T cells, target proteins are purified using a nickel column and subjected to SDE-PAGE and Western Blot analysis. Results illustrate distinct bands in a supernatant corresponding to theoretical molecular weights of the sCAR1 (with a molecular weight of 28 kilodaltons (KDa)) and the CAR3 (with a molecular weight of 18 KDa) (as shown in A of FIG. 2). Subsequent, Western Blot specificity validation is performed by using CAR antibodies and His antibodies, and experimental results illustrate that proteins at these bands are the target proteins of the recombinant expression in the disclosure (as shown in B of FIG. 2).

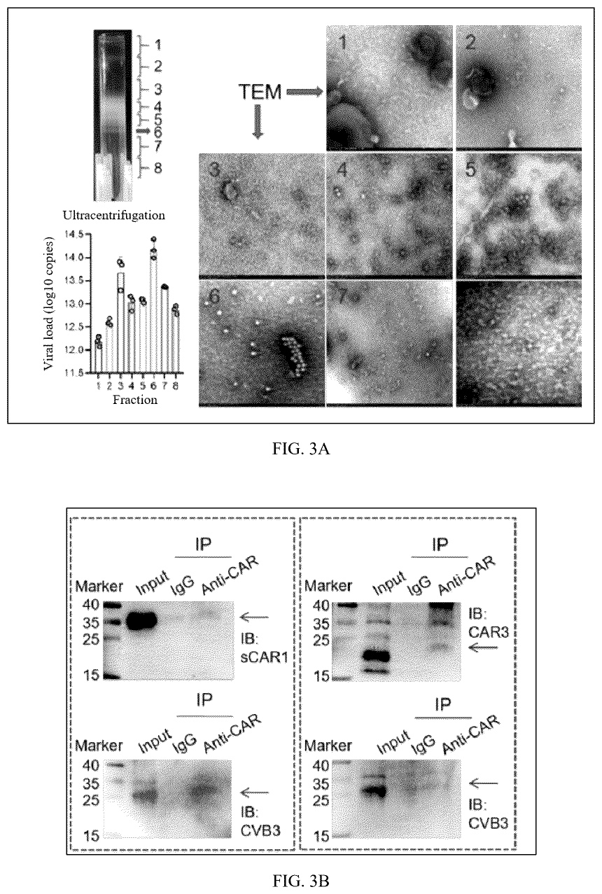

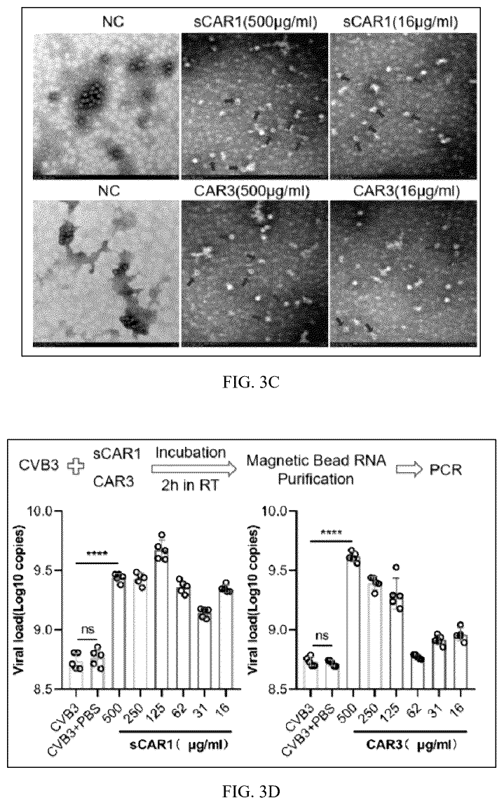

CV-B3 particles (as shown in FIG. 3A) are subjected to an immunoprecipitation assay with sCAR1/CAR3, and results illustrate that the sCAR1/CAR3 exhibits immunoprecipitation with the CV-B3 (as shown in FIG. 3B). This suggests that under ambient, neutral conditions, the sCAR1/CAR3 is capable of binding to the CV-B3. The sCAR1 is capable of inducing a majority of viral solid particles to transform into non-infectious empty capsids, as observed under an electron microscope (as shown in FIG. 3C). Then, a TaqMan probe-based qPCR assay is performed, and results demonstrate that co-incubation of the CV-B3 with varying concentrations of the sCAR1/CAR3, ranging from a high concentration of 500 g/mL to a low concentration of 16 g/mL, significantly promote the release of the viral genome from the CV-B3 into the supernatant (as shown in FIG. 3D).

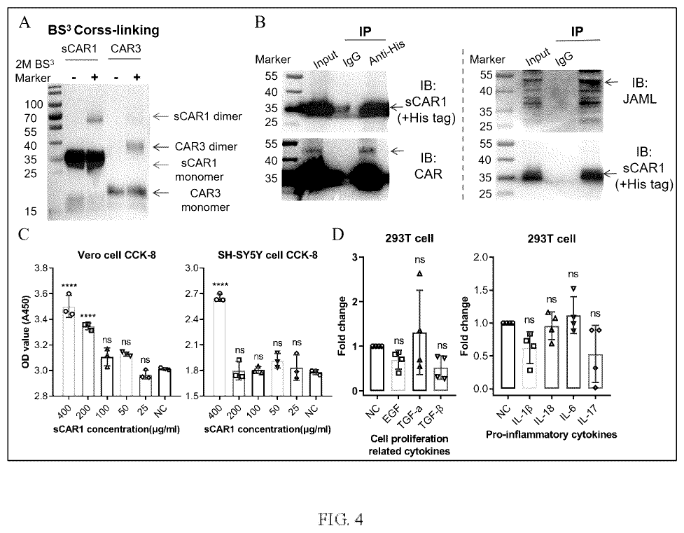

2, sCAR1 Mu does not form intramolecular homodimers and avoids heterodimerization with cellular adhesion molecules, thereby preventing potential abnormal cell proliferation. Specifically, the development of the sCAR1 as a potential molecule against CV-B3 infection must consider its safety and efficacy. However, the formation of intramolecular homodimers after expression of the sCAR1 may affect its binding capacity to the CV-B3 due to certain steric hindrances (as shown in A and B of FIG. 4). In addition, the ability of sCAR1 monomers to bind to cellular membrane surface molecules CAR and JAML may pose safety concerns such as promoting cell proliferation (as shown in C and D of FIG. 4). The disclosure finds that the binding between CAR (UniProt: P97792-1; Gene ID: 1525) and CAR, and between CAR and JAML (UniProt: Q86YT9; Gene ID: 120425), primarily involves ionic bonds formed by amino acids including aspartic acid at position 54 (D54), glutamic acid at position 56 (E56), lysine at position 121 (K121), and hydrogen bonds formed by valine at position 70 (V70), leucine at position 73 (L73), tyrosine at position 83 (Y83), and tyrosine at position 84 (Y84), while the binding of the CAR to the CV-B3 (GenBank accession: PQ001506.1) mainly occurs through hydrogen bonds and ionic bonds formed by threonine at position 24 (T24), glutamic acid at position 56 (E26), threonine at position 44 (T44), and glutamine at position 50 (Q50) with viral particles VP1, VP2, and VP3. Mutations are introduced to the sCAR1/CAR3 to retain amino acid residues involved in strong interactions with the CV-B3, while introducing nonsense mutations at sites involved in strong interactions with CAR itself and JAML (mutating amino acids involved in ionic and hydrogen bonds to alanine).

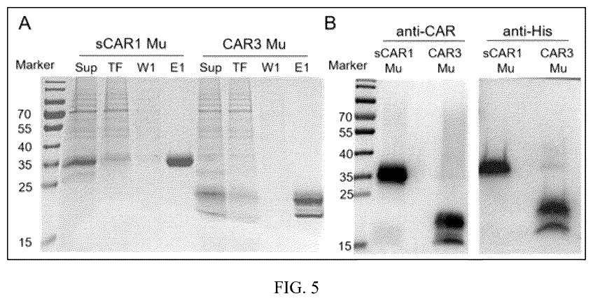

sCAR1 Mu and CAR3 Mu are constructed by the disclosure and are performed plasmid construction, expression, purification, and specificity validation. These results initially demonstrate that introducing mutations at 9 amino acid sites into the sCAR1 and the CAR3 does not affect their normal expression and purification (as shown in A and B of FIG. 5).

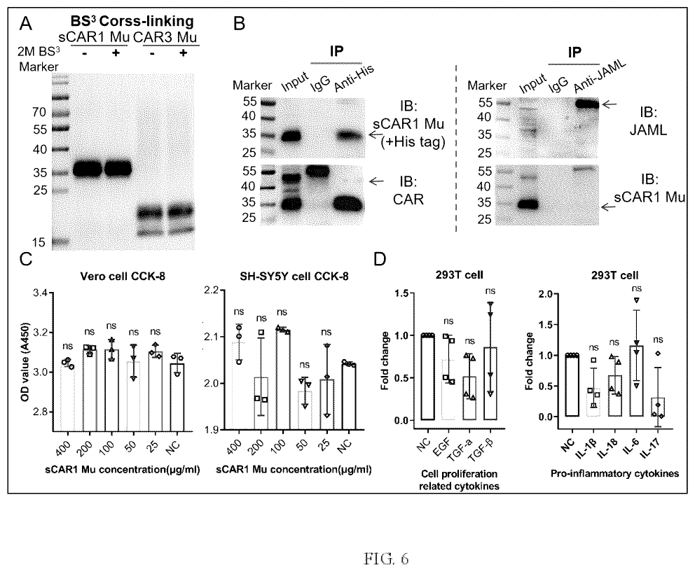

To determine whether the introduction of nonsense mutations at 9 amino acid sites affects the dimerization of recombinant proteins, which is previously established to be formed through ionic and hydrogen bonds, the disclosure directly detects the dimerization of the sCAR1 Mu and the CAR3 Mu under the influence of a BS³ crosslinking agent. Experimental results illustrate bands only at their respective monomeric theoretical molecular weights, indicating that neither the sCAR1 Mu nor the CAR3 Mu form homodimers (as shown in A of FIG. 6), existing solely in monomeric form. Further observation of whether the sCAR1 Mu can bind to the cellular membrane surface molecules CAR and JAML demonstrates that the sCAR1 Mu with a molecular weight of 28 kDa does not undergo immunoprecipitation with the CAR with a molecular weight of 46 kDa and JMAL with a molecular weight of 54 kDa (as shown in B of FIG. 6), suggesting that the introduction of the mutations at 9 amino acid sites eliminate the binding of the sCAR1 Mu to the cellular membrane surface molecules CAR and JMAL. CCK8 assay results indicate that the addition of the sCAR1 Mu at concentrations ranging from low (25 g/mL) to high (400 g/mL) does not exhibit cytotoxicity to Vero cells and SH-SY5Y cells, nor does it significantly affect cell proliferation (as shown in C and D of FIG. 6).

3, Ferritin Particle Display of the sCAR1 Mu Against CV-B3 Activity

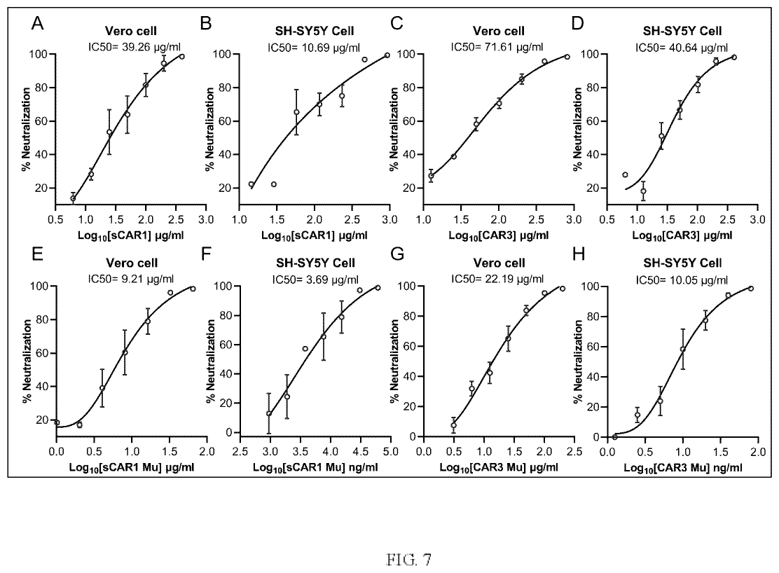

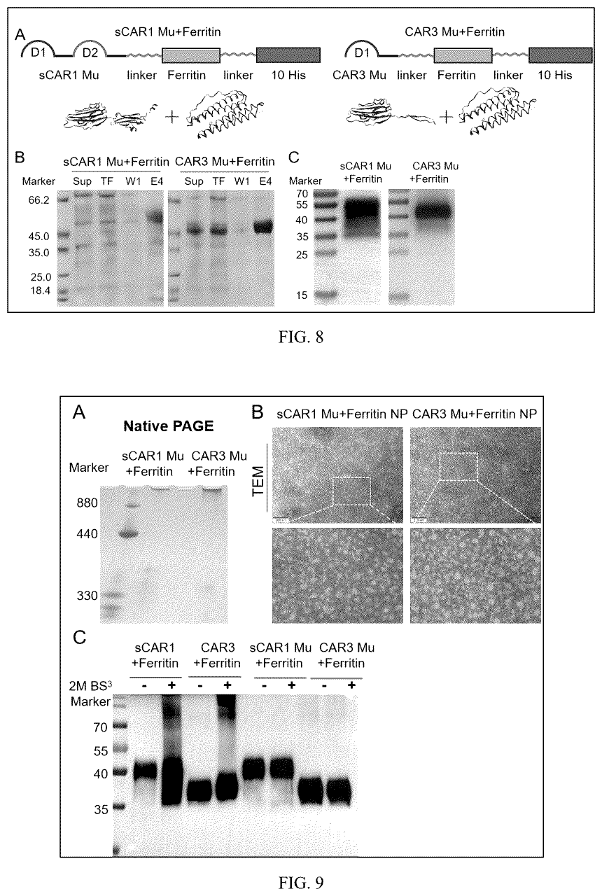

Through the previous site-directed mutagenesis experiments, the sCAR1 Mu and the CAR3 Mu obtained in the disclosure show enhanced safety and efficacy (as shown in FIG. 7), which bolster confidence in further exploring their potential against the CV-B3 infection. To further improve their efficacy and to facilitate subsequent evaluation in animal models, the disclosure leverages Ferritin, a nanoparticle with high safety, strong display capability, and certain delivery properties, to present the sCAR1 Mu and the CAR3 Mu on its surface. Accordingly, sCAR1 Mu-4GS linker-human ferritin heavy chain-10His and CAR3 Mu-4GS linker-human ferritin heavy chain-10His plasmids are constructed (as shown in A of FIG. 8) and transfected into eukaryotic cells for expression and purification, followed by evaluation of their activity against CV-B3 infection (as show in B and C of FIG. 8).

Due to the Ferritin can self-assemble into nanoparticles including 24 subunits (with a theoretical molecular weight of 450 kDa), a native PAGE experiment is performed on sCAR1 Mu+Ferritin and CAR3 Mu+Ferritin. Coomassie blue staining results show bands that correspond to theoretical molecular weights of the sCAR1 Mu+Ferritin (a molecular weight of 1650 kDa) and the CAR3 Mu+Ferritin (a molecular weight of 1410 kDa) (as shown in A of FIG. 9), indicating that the recombinant fusion proteins of the disclosure can assemble into nanoparticles. Uniform particles with a diameter of approximately 15 nm can be observed by using a transmission electron microscope (TEM), and the presence of protrusions on surfaces of these particles indicates the display of the sCAR1 Mu and the CAR3 Mu on surfaces of the Ferritin nanoparticles (as shown in B of FIG. 9). In addition, a BS³ cross-linking experiment is performed and demonstrates that the sCAR1 Mu+Ferritin and the CAR3 Mu+Ferritin do not form dimers (as shown in C of FIG. 9).

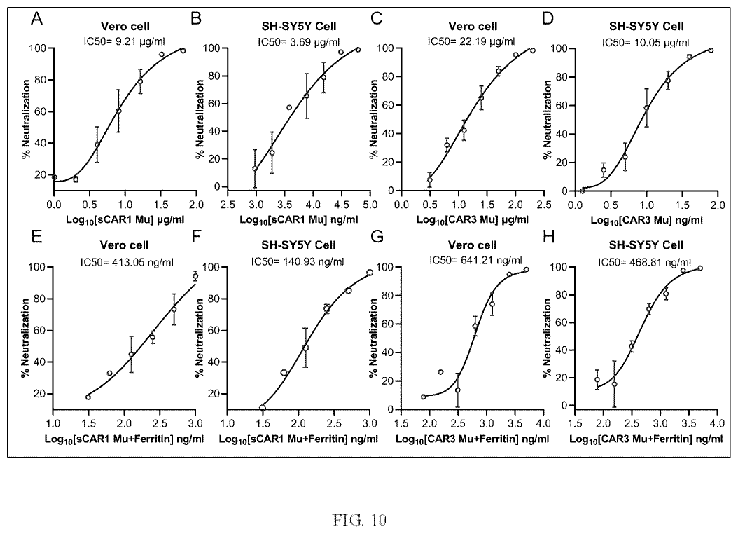

Quantitative polymerase chain reaction (qPCR) using TaqMan probes is performed to measure viral load, and results illustrate that the sCAR1 Mu+Ferritin inhibited CV-B3 infection in Vero cells and SH-SY5Y cells with IC₅₀ values of 413.05 ng/mL and 140.93 ng/mL, respectively (as shown in FIG. 10). Compared to the monomeric sCAR1 Mu, the IC₅₀ values of sCAR1 Mu+Ferritin in Vero cells and SH-SY5Y cells are increased by 22.3-fold and 26.2-fold, respectively.

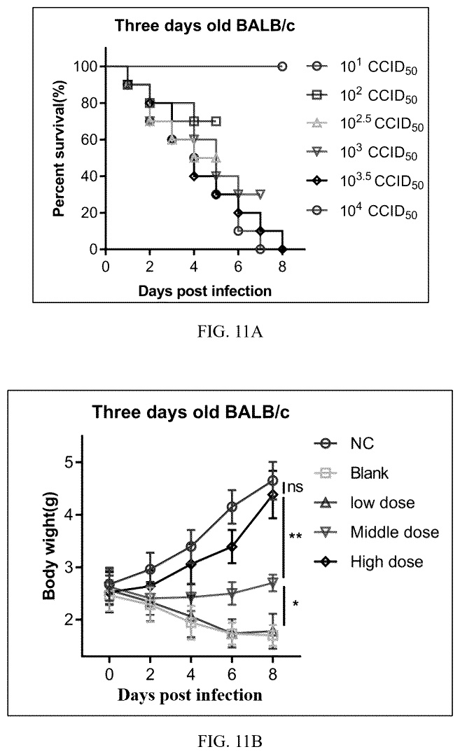

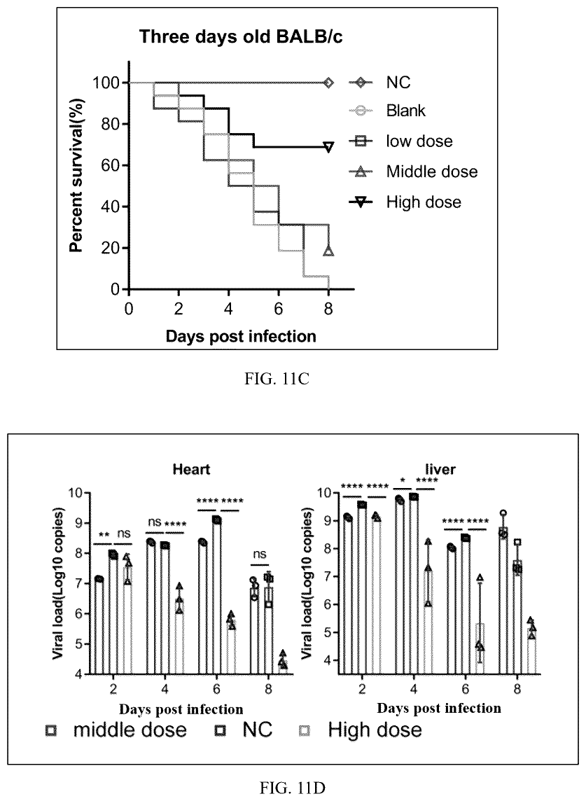

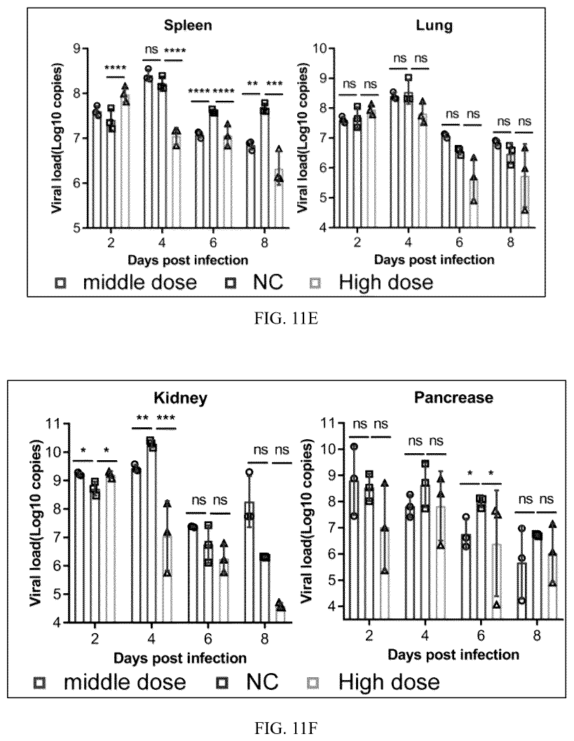

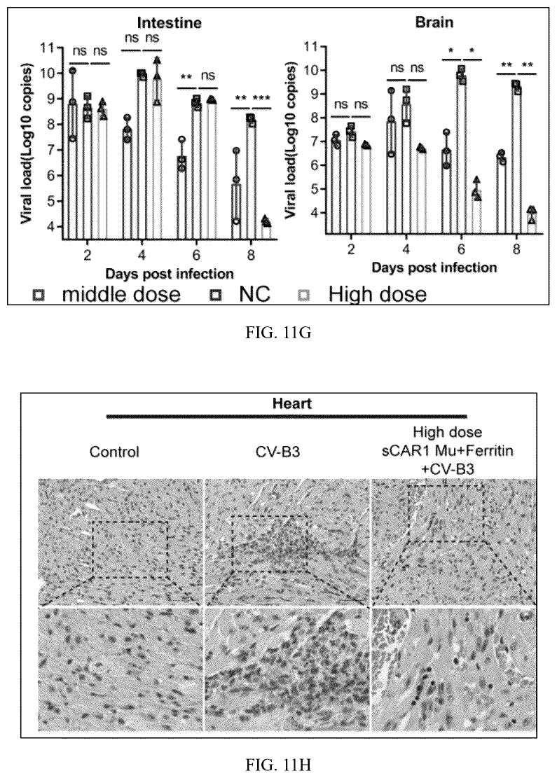

4, sCAR1 Mu+Ferritin can effectively ameliorate disease outcomes and tissue inflammatory pathology in mice following CV-B3 infection. Specifically, mice are divided into different dosage groups and are intraperitoneally injected with high (200 g), medium (100 g), and low (50 g) doses of sCAR1 Mu+Ferritin 6 hours in advanced, followed by an intraperitoneal injection of a 100% lethal dose of CV-B3 (i.e., a CCID₅₀ of 10^{3.5}). Weight changes and survival rates are first observed (as shown in FIG. 11A, FIG. 11B, and FIG. 11C). The disclosure finds that weight changes in the high-dose injection group (200 g) are similar to those in the NC group, and weight continues to increase. The medium-dose injection group (100 g) shows a slow increase in weight, while the low-dose injection group (50 g) and the Blank group exhibit continuous weight loss after intraperitoneal injection of the CV-B3. In terms of survival rate statistics, survival rates of the high, medium, and low-dose injection groups are 68.75%, 18.75%, and 0%, respectively. From the weight and survival rate data, it can be seen that high and medium doses of sCAR1 Mu+Ferritin intervention have a certain protective effect on mice infected with a lethal dose of CV-B3, while the low-dose group did not show a protective effect. In terms of pathological evaluation, the high-dose group can effectively alleviate inflammatory pathology in heart and brain tissues (as shown in FIG. 11A through FIG. 11I).

### 5, Exploration of Expression and Purification Conditions for CAR Protein Analogs

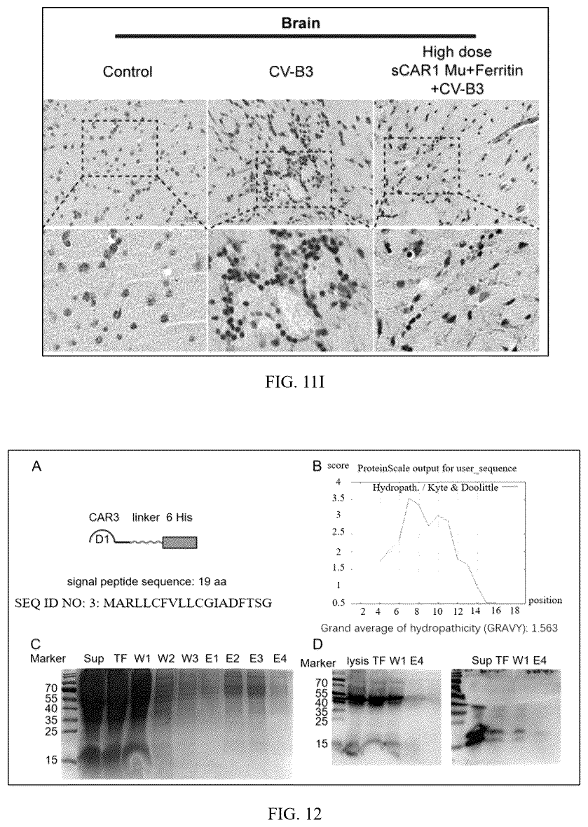

To achieve more effective expression and purification of the CAR protein analogs, the disclosure explores relevant expression and purification conditions by using a CAR3-pTT5 plasmid. As shown in A and B of FIG. 12, the C-terminus of an entire CAR3 sequence (including its own signal peptide) is connected to a 6His purification tag via a 4GS flexible linker and then loaded into a pTT5 vector for expression and purification. The disclosure finds that expressed target protein CAR3 can be detected in both a cell lysate and a supernatant (as shown in D of FIG. 12), but the purification efficiency is very low (as shown in C and D of FIG. 12).

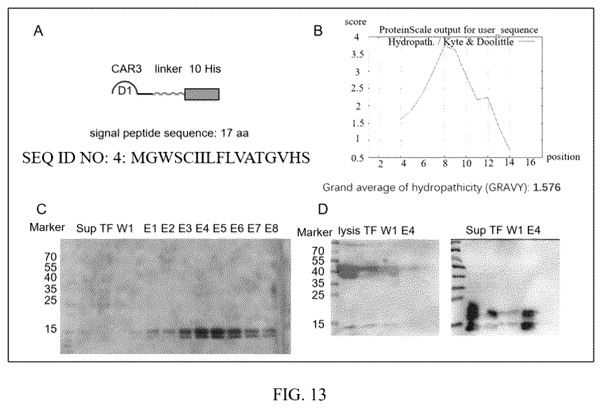

Due to the unsatisfactory purification efficiency when using the 6His purification tag, the disclosure replaces the 6 His tag with a 10 His tag to improve the binding capacity of the expressed target protein to the nickel column (as shown in A of FIG. 13). At the same time, the disclosure finds that when the expressed target protein carries its own signal peptide, it is distributed both inside the cells and in the supernatant. The target protein in the cells requires complex processes such as non-denaturing lysis and ultrasonic disruption for release and extraction. To simplify the extraction process of the target protein and improve its purification efficiency, the disclosure replaces the target protein's own signal peptide (MARLLCFVLLCGIADFTSG, as shown in SEQ ID NO: 3, with a hydrophilicity assessment value of 1.563) with a more hydrophilic signal peptide (MGWSCIILFLVATGVHS, as shown in SEQ ID NO: 4, with a hydrophilicity assessment value of 1.576) (as shown in A and B of FIG. 13), and then carries out expression and purification. During purification, a gradient concentration of imidazole elution is performed (E1: 50 millimoles per liter (mM) imidazole, E2: 100 mM imidazole, E3: 150 mM imidazole, E4: 250 mM imidazole, E5: 300 mM imidazole, E6: 350 mM imidazole, E7: 400 mM imidazole, E8: 450 mM imidazole) to determine an optimal imidazole concentration for efficient elution of the target protein. Purification results (as shown in C of FIG. 13) show that the 10His tag significantly improves purification efficiency, and when the imidazole concentration in the elution buffer reaches 250 mM, the target protein can be effectively eluted. Moreover, the disclosure finds that the target protein with the more hydrophilic signal peptide is predominantly secreted into the supernatant, with minimal intracellular content, resulting in a very low yield after purification (as shown in C and D of FIG. 13). Therefore, based on the above experimental optimization of conditions, in subsequent expression processes of CAR protein analogs, the 10His tag can be selected as the purification tag. The more hydrophilic MGWSCIILFLVATGVHS signal peptide (as shown in SEQ ID NO: 4) can be used to replace the original signal peptide, allowing the majority of the target protein to be secreted into the supernatant, and the elution buffer for the target protein can directly use the imidazole concentration of 250 mM.

As demonstrated by the above embodiments, the nanoparticle provided by the disclosure have the following advantages. 1. The nanoparticle possesses high antiviral activity and can significantly reduce intracellular viral load. 2. The nanoparticle exhibits good safety, is non-toxic to cells, and has no effect on cell proliferation. 3. The nanoparticle possesses strong stability and is easy to store and transport. 4. The nanoparticle improve display efficiency and delivery capacity of the recombinant protein through the Ferritin display system. 5. The nanoparticle are applicable to various cell types, including Vero cells and SH-SY5Y cells, demonstrating a broad antiviral spectrum. 6. The nanoparticle demonstrates protective effects in animal models, effectively alleviating pathological changes caused by viral infections.

In summary, the sCAR1 Mu+Ferritin nanoparticle of the disclosure not only demonstrates potent activity of against the CV-B3 in vitro experiments but also shows favorable protective effects in animal experiments, thereby providing a novel strategy and tool for antiviral therapy.

The embodiments described above are merely specific embodiments of the disclosure. It should be noted that for those skilled in the art, various modifications and refinements can be made to the disclosure without departing from the principles of the disclosure. These modifications and refinements should also be considered within the scope of protection of the disclosure.

What is claimed is:

1. A modified soluble coxsackievirus and adenovirus receptor 1 mutant (sCAR1 Mu) protein, wherein the modified sCAR1 Mu protein is obtained by modifying a soluble coxsackievirus and adenovirus receptor 1 (sCAR1) protein with the amino acid sequence as shown in SEQ ID NO: 2; and
wherein the amino acid sequence of the modified sCAR1 Mu protein is shown in SEQ ID NO: 5.

2. A mutant nanoparticle, wherein the mutant nanoparticle has a ferritin nanoparticle as a matrix and the modified sCAR1 Mu protein as claimed in claim 1 displayed on a surface of the ferritin nanoparticle.

3. The mutant nanoparticle as claimed in claim 2, wherein the amino acid sequence of the mutant nanoparticle is shown in SEQ ID NO: 1.

4. A recombinant expression vector for expressing the mutant nanoparticle as claimed in claim 3, wherein a base vector of the recombinant expression vector is pTT5.

5. An application of the modified sCAR1 Mu protein as claimed in claim 1, comprising:
preparing a drug against Coxsackievirus B3 (CV-B3) by using the modified sCAR1 Mu protein.

6. An application of the mutant nanoparticle as claimed in claim 2, comprising:
preparing a drug against CV-B3 by using the mutant nanoparticle.

7. An application of the mutant nanoparticle as claimed in claim 3, comprising:
preparing a drug against CV-B3 by using the mutant nanoparticle.

8. An application of the recombinant expression vector as claimed in claim 4, comprising:
preparing a drug against CV-B3 by using the recombinant expression vector.

9. A modified coxsackievirus and adenovirus receptor 3 mutant (CAR3 Mu) protein, wherein the modified CAR3 Mu protein is obtained by modifying a coxsackievirus and adenovirus receptor (CAR) protein, and the amino acid sequence of the CAR3 Mu protein is shown in SEQ ID NO: 6.

10. A mutant nanoparticle, wherein the mutant nanoparticle has a ferritin nanoparticle as a matrix and the modified CAR3 Mu protein as claimed in claim 9 displayed on a surface of the ferritin nanoparticle.

11. A mutant nanoparticle, wherein the amino acid sequence of the mutant nanoparticle is shown in SEQ ID NO: 7.

12. An application of the modified CAR3 Mu protein as claimed in claim 9, comprising:
preparing a drug against CV-B3 by using the modified CAR3 Mu protein.

13. An application of the mutant nanoparticle as claimed in claim 10, comprising:
preparing a drug against CV-B3 by using the mutant nanoparticle.

14. An application of the mutant nanoparticle as claimed in claim 11, comprising:
preparing a drug against CV-B3 by using the mutant nanoparticle.


SEQUENCE LISTING (7 sequences)

SEQ ID NO: 1 - protein, 452 aa, synthetic construct
MGWSCIILFLVATGVHSLSITTPEQRIEKAKGETAYLPCKFTLSPEDQGPLAIAWLISPSDNQIVDQAIIAYSGDKIYDNAAPDLKGRVHFTSNDVKSGDASIQVTNLQLSDIGTYQCAVKKAPGVANKKFLLTVLVKPSGTRCFVDGSEEIGNDFKLKCEPKEGSLPLQFEWQKLSDSQTMPTPWLAEMTSPVISVKQASSEYSGTYSCTVQNRVGSDQCMLRLDVVPGGGGSGGGGSGGGGSMTTASTSQVRQNYHQDSEAAINRQINLELYASYVYLSMSYYFDRDDVALKNFAKYFLHQSHEEREHAEKLMKLQNQRGGRIFLQDIKKPDCDDWESGLNAMECALHLEKNVNQSLLELHKLATDKNDPHLCDFIETHYLNEQVKAIKELGDHVTNLRKMGAPESGLAEYLFDKHTLGDSDNESGGGGSGGGGSGGGGSHHHHHHHHHH

SEQ ID NO: 2 - protein, 231 aa, synthetic construct
MARLLCFVLLCGIADFTSGLSITTPEQRIEKAKGETAYLPCKFTLSPEDQGPLDIEWLISPSDNQIVDQVIILYSGDKIYDNYYPDLKGRVHFTSNDVKSGDASINVTNLQLSDIGTYQCKVKKAPGVANKKFLLTVLVKPSGTRCFVDGSEEIGNDFKLKCEPKEGSLPLQFEWQKLSDSQTMPTPWLAEMTSPVISVKNASSEYSGTYSCTVQNRVGSDQCMLRLDVVP

SEQ ID NO: 3 - protein, 19 aa, synthetic construct
MARLLCFVLLCGIADFTSG

SEQ ID NO: 4 - protein, 17 aa, synthetic construct
MGWSCIILFLVATGVHS

SEQ ID NO: 5 - protein, 229 aa, synthetic construct
MGWSCIILFLVATGVHSLSITTPEQRIEKAKGETAYLPCKFTLSPEDQGPLAIAWLISPSDNQIVDQAIIAYSGDKIYDNAAPDLKGRVHFTSNDVKSGDASIQVTNLQLSDIGTYQCAVKKAPGVANKKFLLTVLVKPSGTRCFVDGSEEIGNDFKLKCEPKEGSLPLQFEWQKLSDSQTMPTPWLAEMTSPVISVKQASSEYSGTYSCTVQNRVGSDQCMLRLDVVP

SEQ ID NO: 6 - protein, 162 aa, synthetic construct
MGWSCIILFLVATGVHSLSITTPEQRIEKAKGETAYLPCKFTLSPEDQGPLAIAWLISPSDNQIVDQAIIAYSGDKIYDNAAPDLKGRVHFTSNDVKSGDASIQVTNLQLSDIGTYQCAVKKAPGVANKKFLLTVLGKSSFLLSTGVEWGGGAELQGGREGG

SEQ ID NO: 7 - protein, 385 aa, synthetic construct
MGWSCIILFLVATGVHSLSITTPEQRIEKAKGETAYLPCKFTLSPEDQGPLAIAWLISPSDNQIVDQAIIAYSGDKIYDNAAPDLKGRVHFTSNDVKSGDASIQVTNLQLSDIGTYQCAVKKAPGVANKKFLLTVLGKSSFLLSTGVEWGGGAELQGGREGGGGGGSGGGGSGGGGSMTTASTSQVRQNYHQDSEAAINRQINLELYASYVYLSMSYYFDRDDVALKNFAKYFLHQSHEEREHAEKLMKLQNQRGGRIFLQDIKKPDCDDWESGLNAMECALHLEKNVNQSLLELHKLATDKNDPHLCDFIETHYLNEQVKAIKELGDHVTNLRKMGAPESGLAEYLFDKHTLGDSDNESGGGGSGGGGSGGGGSHHHHHHHHHH